(19) United States
(12) Patent Application Publication
(10) Pub. No.: US 2016/0175364 A1
(43) Pub. Date: Jun. 23, 2016

(54) TROPHOBLAST DERIVED MICROPARTICLES FOR TREATING ISCHEMIC TISSUE AND WOUNDS

(71) Applicants: RAPPAPORT FAMILY INSTITUTE FOR RESEARCH IN THE MEDICAL SCIENCES, Haifa (IL); RAMBAM HEALTH CORPORATION, Haifa (IL)

(72) Inventors: Anat AHARON, Kiryat Tivon (IL); Lior GEPSTEIN, Haifa (IL); Benjamin BRENNER, Haifa (IL)

(21) Appl. No.: 14/908,249

(22) PCT Filed: Jul. 28, 2014
(86) PCT No.: PCT/IL2014/050685

§ 371 Date: Jan. 28, 2016

(30) Foreign Application Priority Data
Jul. 31, 2013 (IL) 227741

(51) Int. Cl. A61K 35/50; A61K 38/18; A61K 38/19; A61K 39/00

(52) U.S. Cl. CPC A61K 35/50; A61K 39/0008; A61K 38/1808; A61K 38/19

(57) ABSTRACT

The invention relates to pharmaceutical compositions comprising trophoblast derived microparticles for the treatment of damaged tissue, including, ischemic tissue and wounds.

### FIELD OF THE INVENTION

The invention relates to pharmaceutical compositions comprising trophoblast derived microparticles for the treatment of damaged tissue, including, ischemic tissue and wounds.

### BACKGROUND

Wound healing progresses in several successive stages, and involves the coordinated efforts of several cell types including keratinocytes, fibroblasts, endothelial cells, macrophages, and platelets. The migration, infiltration, proliferation, and differentiation of these cells are critical for the formation of new tissue and ultimately wound closure. The process is executed and regulated by a complex signaling network involving numerous growth factors, cytokines and chemokines. Of particular importance are epidermal growth factors (EGF), transforming growth factor beta (TGF-beta), fibroblast growth factors (FGF), vascular endothelial growth factors (VEGF), granulocyte macrophage colony stimulating factors (GM-CSF), platelet-derived growth factors (PDGF), connective tissue growth factors (CTGF), interleukins (IL) and tumor necrosis factor-alpha.

Microparticles (MPs) are membrane vesicles shed from various cellular surfaces upon cell activation or apoptosis. MPs vary in size (0.1-1 μm) and in phospholipid and protein composition depending on the cell origin. Similarly, the MPs expose membrane antigens that are specific to their origin, and contain specific growth factors and a variety of cytokines, coagulation factors and adhesion molecules.

MPs are found in blood of healthy individuals, yet, their levels increase in various diseases, such as cancer, diabetes, and vascular complications. Previously, some of the inventors of the present invention have demonstrated that patients with severe diabetic foot ulcers express high levels of MPs originated from platelet and endothelial cells compared to healthy controls (Tsimerman G, et al. Thromb Haemost. 2011; 106:310-21) In this study it was concluded that there is no beneficial effects from treating ulcer or cardiac tissue with patients' autologous MPs.

Human trophoblast cells constitute the interface between maternal and embryonic vascular systems. Trophoblast cells express non-classical, trophoblast-specific tolerogenic human leukocyte antigen G (HLAG) which includes four isoforms: HLA-G1, -G2, -G3 and -G4 that may be detected by antibodies, such as, anti HLA-G1, -G9, -G11 and HLA-G233. HLAG is responsible for inducing maternal immune tolerance to foreign (paternal) antigens.

Recently, some of the inventors of the present invention have demonstrated that microvesicles of healthy pregnant women express angiogenic proteins and reduced apoptosis due to a reduction in caspase 3/7 activity, increased migration, and induced tube formation. (Shomer E et al. Hypertension. 2013 November; 62(5):893-8.).

US Patent Publication No. 2012/0129183 to Aharon et al. discloses methods for isolating circulating placental derived microparticles and use thereof for diagnosing fetal disorders.

Treating ischemic tissue and wounds, especially non-healing wounds, is costly in terms of time and resources required. The annual cost of treating chronic non-healing wounds in the U.S. is approximately $25 billion. Hence, determining a cost-effective and efficacious treatment path is challenging, but crucial. Currently, patients are treated by three growth factors: PDGF-BB, bFGF, and GM-CSF where only PDGF-BB has successfully completed randomized clinical trials in the Unites States. However, the aforementioned growth factors are rapidly inactivated or degraded upon application.

US Patent Publication No. 2002/0001624 to Braun is directed to a medicinal product for topical use for the promotion of wound healing, which comprises thrombocytes or thrombocyte fragments in lyophilized or deep-frozen state, subjected to virus partitioning and/or virus inactivation prior to being used, where the thrombocytes or fragments thereof contain growth factors and are capable of releasing same.

US Patent Publication No. 2004/0082511 to Watzek relates to a drug composition to be applied topically for promoting the regeneration of tissue, characterized in that it contains microparticles from blood cells and/or tissues which have been purified by differential centrifugation, filtration or affinity chromatography, have been subjected to virus inactivation and/or virus depletion, prepared under sterile conditions, and are provided in freeze-dried or deep-frozen state.

### SUMMARY

The present invention discloses methods for treating damaged tissue based on utilizing cell culture trophoblast derived microparticles. The current invention relates to the surprising discovery that trophoblast derived microparticles which can be generated under in vitro growth conditions have a unique composition beneficial for accelerated wound healing.

There is provided, in accordance with some embodiments, a method for treating damaged tissue, the method comprising administering to a patient, in need thereof, a pharmaceutical composition comprising a therapeutically effective amount of microparticles derived from trophoblast cells.

There is provided, in accordance with other embodiments, a method for cell transplantation comprising: transplanting cells and administering to a patient, in need thereof, a pharmaceutical composition comprising a therapeutically effective amount of microparticles derived from trophoblast cells. According to some embodiments, administering said composition is carried out in parallel, before or after the cell transplantation. Thus, the method of the invention is suitable as a single therapy or co-therapy, for example, co-transplantation of cells in parallel to administering trophoblast-derived microparticles.

According to some embodiments, the damaged tissue is selected from the group consisting of venous insufficiency ulcers, pressure ulcers, diabetic wounds, surgical wounds, infected wounds, burns, traumatic wounds, acute or chronic ischemic cardiac tissues, ischemic or non-ischemic cardiomyopathy and bone fractures. Each possibility is a separate embodiment of the invention.

According to some embodiments, the damaged tissue is a symptom of a disease. According to some embodiments, the disease is selected from the group consisting of: myocardial infarction (MI), acute ischemia, chronic ischemia and heart failure. Each possibility is a separate embodiment of the invention.

According to some embodiments, the damaged tissue is a wound. According to some embodiments, the wound comprise chronic wounds. According to some embodiments, the damaged tissue is ischemic tissue. According to some embodiments the ischemic tissue includes cardiac tissue at varying stages of ischemia. According to some embodiments treating ischemic tissues comprises treating ischemic heart disease.

According to some embodiments, administering the pharmaceutical composition comprises administering via a route of administration selected from the group consisting of: subcutaneous, topical, transdermal, oral, buccal, sublingual, sub-labial, intradermal, intravenous, intra-arterial, intracoronary, intramuscular, direct injection to the cardiac tissue and any combination thereof. Each possibility is a separate embodiment of the invention.

According to some embodiments, the pharmaceutical composition is for an external use.

According to some embodiments, the pharmaceutical composition is for topical use, including, topical application in situ, onto cardiac tissue.

According to some embodiments, the pharmaceutical composition is having a dosage form selected from the group consisting of: cream, ointment, gel, paste, powder, aqueous solution, spray, suspension, dispersion and salve. Each possibility is a separate embodiment of the invention.

According to some embodiment, the pharmaceutical composition further comprises a pharmaceutically acceptable carrier.

According to some embodiments, the trophoblasts comprise early-stage trophoblast cells. According to some embodiments, the early-stage trophoblast cells are isolated from second-trimester placenta.

According to some embodiments, the trophoblast cells comprise an in vitro grown trophoblast cell culture. According to some embodiments, the trophoblast cells consist of a trophoblast cell culture. According to some embodiments, the trophoblast cell culture is cultured under starvation prior to obtaining the microparticles. According to some embodiments, the trophoblast cell culture is cultured under hypoxia prior to obtaining the microparticles. According to some embodiments, the trophoblast cell culture is cultured under hypoxia and starvation prior to obtaining the microparticles. According to some embodiments, the microparticles are obtained from the trophoblast cell culture.

According to some embodiments, the microparticles comprise membrane vesicles.

According to some embodiments, the microparticles comprise non-classical HLAG. According to some embodiments, the non-classical HLAG is selected from the group consisting of four isoforms: HLA-G1, -G2, -G3 and -G4, that can be detected by antibodies, such as, anti-HLAG-1, anti-HLAG-9, anti-HLA-G11, anti-HLA-G233 among other antibodies and a combination thereof. Each possibility is a separate embodiment of the invention. It is understood by the skilled in the art that other HLAGs expressed in trophoblast cells are also encompassed by the invention.

According to some embodiments, the microparticles comprise growth factors. According to some embodiments, the growth factors comprise one or more growth factors selected from the group consisting of: epidermal growth factor, fibroblast growth factor, vascular endothelial growth factor, platelet-derived growth factor, granulocyte macrophage colony stimulating factor, transforming growth factor beta, connective tissue growth factor, placenta growth factor, insulin growth factor and combinations thereof. Each possibility is a separate embodiment of the invention.

According to some embodiments, the microparticles comprise epidermal growth factor.

According to some embodiments, the microparticles comprise at least one cytokine. Additionally or alternatively, the microparticles comprise at least one matrix metalloproteinase (MMP) inhibitor. Additionally or alternatively, the microparticles comprise at least one tissue inhibitor of metalloproteinase (TIMP).

According to some embodiments, the cytokines are selected from interleukin 6, interleukin 8 and a combination thereof. Each possibility is a separate embodiment of the invention.

According to some embodiments, the tissue inhibitors of metalloproteinase are selected from TIMP-1 and TIMP-2 and a combination thereof. Each possibility is a separate embodiment of the invention.

According to some embodiments, treating comprises one or more of inducing endothelial cell migration, inducing tissue vascularization, inducing endothelial cell survival, reducing apoptosis, inducing expression of Bcl-2, decreasing caspase 3/7 activity, and inducing cell graft survival. Each possibility is a separate embodiment of the invention.

According to some embodiments, the microparticles of the pharmaceutical composition are derived from trophoblast cells isolated from the plasma of pregnant subject(s). According to some embodiments, the subject in need thereof is different from said pregnant subject, or one of said pregnant subjects.

According to some embodiments, the subject in need thereof is diabetic.

According to some embodiments, there is provide a method comprising providing a trophoblast cell culture; isolating from said cell culture microparticles; and administering to a patient, in need thereof, a pharmaceutical composition comprising a therapeutically effective amount of said microparticles.

According to some embodiments, prior to isolating the microparticles the trophoblast cell culture is exposed to starvation conditions.

According to some embodiments, prior to isolating the microparticles the trophoblast cell culture is exposed to hypoxia.

There is provided, in accordance with some embodiments, a pharmaceutical composition comprising a therapeutically effective amount of microparticles derived from trophoblast cells for the use in treating damaged tissue.

There is provided, in accordance with some embodiments, a pharmaceutical composition comprising a therapeutically effective amount of microparticles derived from trophoblast cells for the use in the treatment of damaged tissue.

There is provided, in accordance with some embodiments, a first pharmaceutical composition comprising a therapeutically effective amount of microparticles derived from trophoblast cells for the use in the treatment of damaged tissue in combination with a second pharmaceutical composition comprising a therapeutically effective amount of at least one agent selected from the group consisting of antiseptics, antibiotics, anti-viral agents, anti-fungal agents, anti-inflammatory agents, non-antibiotic anti-microbial agents, one or more anti-viral agents and analgesics.

According to some embodiments, the pharmaceutical composition is administered via a route of administration comprising: subcutaneous, topical, transdermal, oral, buccal, sublingual, sub-labial, intradermal, intravenous, intra-arterial, intracoronary, intramuscular, direct injection to the cardiac tissue and any combination thereof. Each possibility is a separate embodiment of the invention.

According to some embodiments, the first or second pharmaceutical compositions are administered via a route of administration comprising: subcutaneous, topical, external, transdermal, oral, buccal, sublingual, sub-labial, intradermal, intravenous, intra-arterial, intracoronary, intramuscular, direct injection to the cardiac tissue and any combination thereof. Each possibility is a separate embodiment of the invention. According to some embodiments, the microparticles of the pharmaceutical composition are derived from trophoblast cells isolated from the plasma of pregnant subjects. According to some embodiments, the pregnant subject is different from the subject in need thereof.

According to some embodiments, the subject in need thereof is diabetic.

According to some embodiments, there is provided a kit for treating damaged tissue, the kit comprising the pharmaceutical composition as described in embodiments herein and instructions for use thereof.

According to some embodiments, there is provided a method of treating damaged tissue in a subject in need thereof, the method comprising administering to the subject a pharmaceutical composition comprising a therapeutically effective amount of microparticles derived from trophoblast cells.

According to some embodiments, the method further comprises administering to said subject antiseptics, antibiotics, anti-viral agents, anti-fungal agents, anti-inflammatory agents, non-antibiotic anti-microbial agents, one or more anti-viral agents, analgesics and a combination thereof.

According to some embodiments, there is provided a combined therapy of a damaged tissue in a subject in need thereof, said combined therapy comprises administering to said subject a first pharmaceutical composition comprising a therapeutically effective amount of microparticles derived from trophoblast cells and a second pharmaceutical composition comprising a therapeutically effective amount of at least one agent selected from the group consisting of antiseptics, antibiotics, anti-viral agents, anti-fungal agents, anti-inflammatory agents, non-antibiotic anti-microbial agents, one or more anti-viral agents and analgesics.

According to some embodiments, the microparticles of the pharmaceutical composition are derived from trophoblast cells isolated from the plasma of pregnant subjects. According to some embodiments, the pregnant subject is different from the subject in need thereof.

According to some embodiments, the subject in need of treatment is diabetic.

According to some embodiments, the damaged tissue is selected from the group consisting of: venous insufficiency ulcers, pressure ulcers, diabetic wounds, surgical wounds, infected wounds, burns, traumatic wounds, acute or chronic ischemic cardiac tissues, ischemic or non-ischemic cardiomyopathy and bone fractures. Each possibility is a separate embodiment of the invention.

According to some embodiments, the damaged tissue is a wound. According to some embodiments, the wound is a chronic wound. According to some embodiments, the damaged tissue is ischemic tissue.

According to some embodiments, the trophoblast cells are early-stage trophoblast cells. According to some embodiments, the trophoblast cells comprise an in vitro grown trophoblast cell culture. According to some embodiments, the trophoblast cell culture is exposed to starvation condition prior to obtaining the microparticles. According to some embodiments, the microparticles are obtained from the trophoblast cell culture.

According to some embodiments, the microparticles comprise membrane vesicles. According to some embodiments, the microparticles comprise non-classical HLAG. According to some embodiments, the non-classical HLAG is selected from HLA-G1, HLA-G9, HLA-G11, HLA-G233 and a combination thereof. Each possibility is a separate embodiment of the invention.

According to some embodiments, the microparticles comprise growth factors. According to some embodiments, the growth factors comprise one or more growth factors selected from the group consisting of: epidermal growth factor, fibroblast growth factor, vascular endothelial growth factor, platelet-derived growth factor, granulocyte macrophage colony stimulating factor, transforming growth factor beta, connective tissue growth factor, placenta growth factor and insulin growth factor. Each possibility is a separate embodiment of the invention. According to some embodiments, the microparticles comprise epidermal growth factor.

According to some embodiments, the microparticles comprise cytokines. According to some embodiments, the cytokines are selected from interleukin 6, interleukin 8 and a combination thereof. Each possibility is a separate embodiment of the invention.

According to some embodiments, treating damaged tissue comprises one or more of inducing endothelial cell migration, inducing tissue vascularization, inducing endothelial cell survival, reducing apoptosis, inducing expression of Bcl-2 and decreasing caspase 3/7 activity. Each possibility is a separate embodiment of the invention.

According to some embodiments, the microparticles are derived from trophoblast cells isolated from the plasma of pregnant subjects. According to some embodiments, the pregnant subject is different from the subject in need thereof.

According to some embodiments, administering the pharmaceutical composition comprises applying said pharmaceutical composition onto said damaged tissue.

According to some embodiments, applying comprises periodically applying the pharmaceutical composition until the damaged tissue heals.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. It is intended that the embodiments and figures disclosed herein are to be considered illustrative rather than restrictive. The figures are listed below.

FIG. 1 shows extra-membrane HLAG molecules distribution in trophoblast cells and in trophoblast derived microparticles as determined by FACS analysis (using antibodies for HLAG-1, HLAG-9, HLA-G11 and HLA-G233);

FIG. 2A shows the protein expression profile of key markers of angiogenic and inflammatory proteins in MPs obtained from healthy pregnant women (HP) as a percentage of that obtained in MPs from non-pregnant women (NP);

FIG. 2B shows the protein expression profile obtained in MPs derived from trophoblast as a percentage of that obtained in MPs derived from a myeloma cell line, as determined by protein array analysis;

FIG. 3A shows non-treated HUVEC migration;

FIG. 3B shows migration of HUVEC, pre-treated with microparticles obtained from healthy pregnant women (MP-HP);

FIG. 3C shows migration of HUVEC, pre-treated with trophoblast cell derived microparticles (HVT-MP);

FIG. 3D shows the quantitative results from cell migration of HUVEC non-treated (No-MPs; n=4) or treated with microparticles obtained from healthy pregnant women (HP-MPs; n=4);

FIG. 3E show the quantitative results from cell migration of HUVEC non-treated (No-MPs; n=6) or treated with trophoblast derived microparticles (HVT-MPs; n=5);

FIG. 4A presents a schematic outline of an in vitro wound healing assay, based on cell migration;

FIG. 4B is a representative image of in vitro wound healing in non-treated HUVEC;

FIG. 4C shows a representative image of in vitro wound healing in HUVEC treated with trophoblast cell derived MPs (HVP-MPs);

FIG. 4D shows the quantitative results of the wound healing assay;

FIG. 5A shows representative images of tube formation in non-treated HUVEC (NT) and in HUVEC treated with microparticles obtained from healthy pregnant women (HP-MPs);

FIG. 5B shows quantitative results of HUVEC tube formation either with non-treated HUVEC (NT; n=21) or following incubation (treatment) of HUVEC with microparticles obtained from healthy pregnant women (HP-MPs; n=20) for 1 h;

FIG. 5C shows quantitative results of HUVEC tube formation either with non-treated HUVEC (NT; n=19) or following incubation (treatment) of HUVEC with microparticles obtained from healthy pregnant women (HP-MPs; n=20) for 2 h;

FIG. 5D shows quantitative results of HUVEC tube formation either with non-treated HUVEC (NT; n=19) or following incubation (treatment) of HUVEC with microparticles obtained from healthy pregnant women (HP-MPs; n=10) for 4 h;

FIG. 6A shows percentage of caspase 3/7 activity in HUVEC (No MPs; n=9) and in HUVEC exposed to microparticles derived from healthy pregnant women (HP-MPs; n=9);

FIG. 6B shows Bcl-2 mRNA expression in HUVEC (No MPs; n=3) and in HUVEC exposed to microparticles derived from healthy pregnant women (HP-MPs; n=8);

FIG. 7 shows the percentage of apoptosis determined by TUNELL assay of rat cardiomyocytes (n=5), starved (n=5) or exposed to 25 μg (n=7) or 50 μg (n=5) trophoblast derived microparticles;

FIG. 8A shows representative images of wounds of healthy rats treated with trophoblast derived microparticles, as compared to vehicle treated;

FIG. 8B shows the wound area size as a function of time in wounds of healthy rats treated with trophoblast derived microparticles, as compared to vehicle treated;

FIG. 9A shows representative images of wounds of diabetic rats treated with 500 μg trophoblast derived microparticles, as compared to untreated;

FIG. 9B shows the wound area size as a function of time in wounds of diabetic rats treated with 250 μg trophoblast derived microparticles, as compared to untreated;

FIG. 9C shows the wound area as a function of time in wounds of diabetic rats treated with 500 μg trophoblast derived microparticles, as compared to untreated;

FIG. 10A show representative images of CD34 histological staining in wound samples, using an in vivo wound healing model;

FIG. 10B shows a quantification of FIG. 10A;

FIG. 11A show representative images of CD68 histological staining in wound samples, using an in vivo wound healing model;

FIG. 11B shows a quantification of FIG. 11A;

FIG. 12A show representative images of Collagen histological staining in wound samples, using an in vivo wound healing model;

FIG. 12B shows a quantification of FIG. 12A.

### DETAILED DESCRIPTION

The present invention discloses pharmaceutical compositions comprising trophoblast derived microparticles for the treatment of ischemic tissue and/or wounds.

There is provided, in accordance with some embodiments, a method for treating damaged tissue, the method comprising administering to a patient, in need thereof, a pharmaceutical composition comprising a therapeutically effective amount of microparticles derived from trophoblast cells.

There is provided, in accordance with other embodiments, a method for cell transplantation comprising: transplanting cells and administering to a patient, in need thereof, a pharmaceutical composition comprising a therapeutically effective amount of microparticles derived from trophoblast cells. According to some embodiments, administering said composition is carried out in parallel, before or after the cell transplantation. Thus, the method of the invention is suitable as a single therapy or co-therapy, for example, co-transplantation of cells in parallel to administering trophoblast-derived microparticles.

It is to be understood that the methods of the inventions may be applied for treating or preventing cardiovascular disease (CVD). The methods may be applied in patients that already experienced myocardial infarction (MI). In these patients the cardiac tissue is damaged, yet not totally damaged. Thus, the method of the invention is directed at maintaining and even improving the viability of the viable tissue and cells that survived the MI.

Furthermore, the methods of the invention is directed to preventing apoptosis and improve perfusion thereby reduce the acute MI effect and the damage to the cardiac tissue associated therewith.

There is provided, in accordance with other embodiments, a method for treating ischemic heart disease comprising: transplanting cells and administering to a patient, in need thereof, a pharmaceutical composition comprising a therapeutically effective amount of microparticles derived from trophoblast cells. According to some embodiments, administering said composition is carried out in parallel, before or after the cell transplantation. Thus, the method of the invention is suitable as a single therapy or co-therapy, for example, co-transplantation of cells in parallel to administering trophoblast-derived microparticles.

As used herein, the term “ischemic tissue” refers to tissue damage and or tissue scaring resulting from impaired blood flow to the tissue.

According to some embodiments, the method of the invention is for treating ischemic heart disease.

As used herein, the terms “ischemic heart disease” and “IHD” interchangeably refer to a spectrum of disorders ranging from acute and chronic ischemia due to coronary artery disease (resulting in angina pectoris syndromes), to acute myocardial infarction, as well as chronic ischemic cardiomyopathy; the latter is characterized by severe myocardial scarring including multiple sites of replacement fibrosis and diffuse interstitial fibrosis.

Recently, various cell types have been used for potential cell-based therapies to treat both acute and chronic ischemia as well as to reduce cardiac scar size and improve cardiac function in patients with ischemic cardiomyopathy. Without wishing to be bound by any theory or mechanism, combining cell therapy with the method of the invention provides an advantageous treatment.

As used herein, the term “wound” refers to open wounds in which the skin is torn, cut, or punctured, and to closed wounds in which blunt force trauma causes contusions. According to some embodiments, the term “wound” refers broadly to injuries to the skin and subcutaneous tissue as well as internal organs initiated in any one of a variety of ways (e.g., wounds afflicted by an infectious organism) and with varying characteristics. Exemplary examples include, but are not limited to, bruises, scrapes, burn wounds, sunburn wounds, incisional wounds, excisional wounds, surgical wounds, necrotizing fascitis, ulcers, venous stasis ulcers, vascular ulcers, diabetic ulcers, decubitus ulcers, aphthous ulcers, pressure ulcers, lesions, scars, alopecia areata, dermatitis, allergic contact dermatitis, atopic dermatitis (dyshidrotic eczema), colitis, berloque dermatitis, diaper dermatitis, dyshidrotic dermatitis (Pompholyx), psoriasis, eczema, erythema, warts, anal warts, angioma, cherry angioma, athlete's foot, atypical moles (Clark's nevus), basal cell carcinoma, Bateman's purpura, bullous pemphigoid, Candida, chondrodermatitis helicis, cold sores, condylomata, cysts, Darier's disease, dermatofibroma, Discoid Lupus Erythematosus (Lupus of the Skin), nummular eczema, atopic eczema, hand eczema, Multiforme Erythema Nodosum, Fordyce's Condition, Folliculitis Keloidalis Nuchae, Folliculitis, Granuloma Annulare, Grover's Disease, heat rash, herpes simplex, herpes zoster (shingles), Hidradenitis Suppurativa, Hives, Hyperhidrosis, Ichthyosis, Impetigo, Keratosis Pilaris, Keloids, Keratoacanthoma, Lichen Planus, Lichen Planus Like Keratosis, Lichen Simplex Chronicus, Lichen Sclerosus, Lymphomatoid Papulosis, skin disorders associated with Lyme Disease, Lichen Striatus, Myxoid Cysts, Mycosis Fungoides, Molluscum Contagiosum, Moles, Nail Fungus (Onychomycosis), Necrobiosis Lipoidica Diabeticorum, Nummular Dermatitis, Onychoschizia, Pityriasis Lichenoides, Pityriasis Rosea, Pityriasis Rubra Pilaris, plantar warts, Poison Ivy rash, Poison Oak rash, Pseudofolliculitis barbae, Pruritus Ani and Pityriasis Alba. Each possibility is a separate embodiment.

The term “vascular ulcers” as used herein refers to peripheral vascular disease (PVD) which is a nearly pandemic condition which may in some instances require limb amputation and even cause death of the patient. PVD manifests as insufficient tissue perfusion caused by existing atherosclerosis together with either emboli or thrombi. Ulceration due to inadequate vascularization is multifactorial and can be related to arterial and to venous diseases.

The term “diabetic ulcer” relates to ulcers in patients having diabetes mellitus (DM)-type 1 (insulin-dependent) or DM type 2 (non-insulin dependent) which are associated with vascular complications. Lower extremity infections (mainly foot infections) are the most common reason for hospital admission among the diabetic population.

As used herein, the terms “non-healing wounds” and “chronic wounds” interchangeably refer to wounds that fail to heal. In some embodiments, non-healing wounds include, but are not limited to, wounds which fail to heal in an orderly and timely manner. In some embodiments, non-healing wounds refer to wounds which remain un-healed for more than 20 days, a month, two months, three months or more.

As used herein the term “wound healing” refers to a multistage process. In stage I, the blood plasma protein fibrinogen is precipitated by thrombin so as to induce the formation of fibrin clots, which solidifies in the presence of blood coagulation factor XIII. The first stage takes only minutes during which bleeding is controlled and the wound area sealed. In stage II, cells from the wound area (i.e. inflammatory cells, connective tissue cells and endothelial cells) migrate into the fibrin clot. They form vessels and connective tissue primarily comprised of collagen. This connective tissue, which is referred to as granulation tissue, serves as the substratum for the formation of epithelial tissue and is the substratum for the epidermis on the body surface. This stage is characterized by high expression of the endothelial cell marker CD34 and the neutrophil cell marker CD68. Stage II lasts for days to weeks and is complete when the wound area has been closed by epithelium, and by the epidermis on the skin. This stage is characterized with enhanced collagen density.

As used herein the terms “microparticles” and “MPs” interchangeably refer to membrane vesicles shed from various cellular surfaces upon cell activation or apoptosis.

According to some embodiments, administering the pharmaceutical composition comprises administering via a route of administration selected from the group consisting of: subcutaneous, topical, transdermal, oral, buccal, sublingual, sub-labial, intradermal and any combination thereof. Each possibility is a separate embodiment of the invention.

According to some embodiments, the pharmaceutical composition is for an external use.

According to some embodiments, the pharmaceutical composition is a composition for an external use having a dosage form selected from the group consisting of: cream, ointment, gel, paste, powder, aqueous solution, spray, suspension, dispersion and salve. Each possibility is a separate embodiment of the invention.

According to some embodiment, the pharmaceutical composition further comprises a pharmaceutically acceptable carrier.

The term “pharmaceutically acceptable carrier” is well known in the art and includes, but is not limited to, carriers such as saline, buffered saline, dextrose, water, glycerol, sterile isotonic aqueous buffer and combinations thereof. One example of such an acceptable carrier is a physiologically balanced culture medium containing one or more stabilizing agents such as stabilized, hydrolyzed proteins, lactose, etc. The carrier is preferably sterile and is adapted to suit the mode of administration.

According to some embodiments, the damaged tissue is selected from the group consisting of venous insufficiency ulcers, pressure ulcers, diabetic wounds, surgical wounds, infected wounds, burns, traumatic wounds, ischemic cardiomyopathy, bone fractures and damaged tissue associated with dermal anti-aging treatments. Each possibility is a separate embodiment of the invention.

According to some embodiments, the damaged tissue are wounds. According to some embodiments, the wounds comprise chronic wounds. According to some embodiments, the damaged tissue is ischemic tissue. According to some embodiments treating ischemic tissues comprise treating ischemic heart disease.

According to some embodiments, the trophoblast cells comprise early-stage trophoblast cells.

As used herein, the term “early-stage trophoblast cells” refers to trophoblast cells isolated from second-trimester placenta.

According to some embodiments, the trophoblast cells are grown in cell culture in vitro. Hence it is understood by the skilled in the art that the microparticles can be obtained from trophoblast cells grown in cell culture. The trophoblast cells can advantageously be purchased and do not have to be freshly isolated trophoblast cells. Hence, the trophoblast cell culture provides both: a relatively inexpensive source and an easily obtained source, of microparticles. Alternatively, the microparticles may be derived from freshly isolated trophoblast cells. According to some embodiments, the microparticles are isolated and purified microparticles.

According to some embodiments, the trophoblast cell culture is exposed to stress conditions prior to obtaining the microparticles. Exemplary stress conditions are starvation and hypoxia. It is however understood by the skilled in the art that additional treatments of the trophoblast cells or culture conditions, which render the trophoblast cells more prone to produce microparticles are also covered by the present invention.

According to some embodiments, starvation conditions include, but are not limited to, culture in a culture medium devoid of serum, culture in a culture medium free of growth factor, amino acids, vitamins, cytokines and/or hormones. According to some embodiments, the trophoblast cells are cultured under starvation or hypoxia conditions. According to some embodiments, the trophoblast cells are cultured under starvation or hypoxia conditions for at least 12 hrs. According to some embodiments, the trophoblast cells are cultured under starvation or hypoxia conditions for at least 24 hours. According to some embodiments, the trophoblast cells are cultured under starvation or hypoxia conditions for at least 48 hours.

According to some embodiments, the microparticles comprise non-classical HLAG. According to some embodiments, the non-classical HLAG is selected from the group consisting of: HLA-G1, -G2, -G3 and -G4 molecules, that may be detected by antibodies such as anti-HLAG-1, anti-HLAG-2, anti-HLAG-3, anti-HLAG-4, anti-HLAG-9, anti-HLA-G11, anti-HLA-G233 and any combination thereof. Each possibility is a separate embodiment of the invention.

According to some embodiments, the microparticles do not induce an immune response. Without being bound by any theory, the non-classical HLAG molecules induce maternal immune tolerance toward the fetus via the interaction of the non-classical HLAG molecules with inhibitory receptors on maternal NK cells and CD8+T lymphocytes.

According to some embodiments, the microparticles comprise growth factors. According to some embodiments, the growth factors comprise one or more growth factors selected from the group consisting of: epidermal growth factor (EGF), fibroblast growth factor (FGF), vascular endothelial growth factor (VEGF), platelet-derived growth factor (PDGF), granulocyte macrophage colony stimulating factor (GM-CSF), transforming growth factor beta (TGF-beta), connective tissue growth factor (CTGF), placenta growth factor (PLGF), insulin growth factor (IGF) or combinations thereof. Each possibility is a separate embodiment of the invention.

According to some embodiments, there is provided a kit for treating damaged tissue, the kit comprising the pharmaceutical composition comprising a therapeutically effective amount of microparticles derived from trophoblast cells, as described in embodiments herein and instructions for use thereof.

According to some embodiments, the kit further comprises antiseptics, antibiotics and a combination thereof.

According to some embodiments, the kit further comprises protecting means. According to some embodiments, said protecting means are selected from a bandage, a compress, a band aid, a dressing and a combination thereof. Each possibility is a separate embodiment of the invention.

According to some embodiments, there is provided a method of treating damaged tissue in a subject in need thereof, the method comprising administering to the subject a pharmaceutical composition comprising a therapeutically effective amount of microparticles derived from trophoblast cells, as described in embodiments herein.

According to some embodiments, treating the subject in need thereof may include treating the damaged tissue of the subject with microparticles derived from a trophoblast cell culture. Additionally or alternatively, treating the subject in need thereof may include treating the damaged tissue of the subject with microparticles obtained from the plasma of healthy pregnant subjects. It is understood, that the pregnant subject may be different from the subject in need of the treatment.

According to some embodiments, the subject in need of treatment is diabetic. As used herein the term “diabetic” refers to subjects suffering from type I and/or type II diabetes. According to some embodiments, the diabetic subject suffer from chronic wounds and/or ulcers.

According to some embodiments, administering the pharmaceutical composition comprises topical administration onto the damaged skin, the healthy skin at the periphery of the damaged skin and a combination thereof. According to some embodiments, administering the pharmaceutical composition comprises applying the pharmaceutical composition onto the damaged tissue. As used herein the term “applying” may refer to swabbing, smearing, dripping or otherwise causing the pharmaceutical composition to be absorbed by the wound subject to treatment. Each possibility is a separate embodiment of the invention.

According to some embodiments, applying the pharmaceutical composition comprises periodically applying said pharmaceutical composition for example until the damaged tissue heals. As used herein the term periodically may include to applying the composition every 2 hours, every 4 hours, every twelve hours, daily, every two days, every 3 days, once a week or according to any other suitable treatment regimen within the described ranges or other ranges. Each possibility is a separate embodiment of the invention.

As used herein, the term “damaged tissue heals” means that the damage in the tissue is not further progressing, or that healing process are in development or in progress. Each possibility is a separate embodiment of the invention.

Improvement includes reduction in size, reduction in severity, reduction is pain and a combination thereof.

As illustrated herein below, the trophoblast derived MPs contain significantly elevated epidermal growth factor (EGF) levels, PLGF, Angiopoietins land 2, and other growth factors as well as tissue inhibitors of metalloproteinases (TIMPs), as compared to MPs of alternative cell origin such as MPs derived from the plasma of healthy pregnant women or MPs derived from a myeloma cell line.

According to some embodiments, the microparticles comprise cytokines. According to some embodiments, the cytokines are selected from interleukin 6, interleukin 8 and a combination thereof. Each possibility is a separate embodiment of the invention.

A common difficulty in cell transplantation, is that most of the cells undergo apoptosis or necrosis. The method of the invention can improve transplantation if applied in parallel with transplantations. Thus, according to some embodiments, there is provided a method for transplanting cells in a recipient, the method comprising the steps of: (a) transplanting the cells into the recipient; and (b) administering to the recipient a dose of a pharmaceutical composition comprising a therapeutically effective amount of microparticles derived from trophoblast cells.

As illustrated herein below, the trophoblast derived MPs contain significantly elevated levels of IL6 and IL8, as compared to MPs of alternative cell origin such as MPs derived from the plasma of healthy pregnant women or MPs derived from a myeloma cell line.

As further illustrated herein below, the trophoblast derived microparticles induce endothelial cell migration, tissue vascularization, tissue perfusion, provide for maintenance of capillaries and/or endothelial cell survival, as well as reduced cell apoptosis and improved cell viability.

In addition to the exemplary aspects and embodiments described above, further aspects and embodiments will become apparent by reference to the figures and by study of the following examples. The materials, methods, and examples discussed below are illustrative and are not intended to limit the scope of the invention.

### EXAMPLES

### Example 1

### Material and Methods

MP Isolation from Blood of Healthy Pregnant Women (HP-MPs):

15 ml of peripheral venous blood was drawn from healthy pregnant women into sodium citrate (3.2%) tubes; platelet-poor-plasma (PPP) was prepared after two centrifugations (10 min, 1500 g) within an hour from collection and frozen at −80° C. MPs were isolated from thawed PPP by centrifugation (1 h, 18,000 g).

MP Isolation from Trophoblast Cells (HVT-MPs):

Early stage trophoblast (EST) cells isolated from second trimester placenta were purchased from ScienCell (Carlsbad, Calif., USA). The cells were re-cultured in modified medium containing 50% trophoblast medium with supplements (as provided by ScienCell), 22% DMEM, 22% F12, 4% fetal calf serum (FCS), 1% antibiotics (10,000 units/ml penicillin, 10 mg/ml streptomycin, 250 units/ml nyastatin), 0.0001% amphotericin B. Cells were incubated at 37° C., 5% CO₂ and used for experiments at passages 4-15.

When the trophoblast cells culture reached ˜80% confluence, the cells were cultured under starvation, namely, in a medium free of serum or any supplements of growth factors. Alternatively, the trophoblast cells were cultured under hypoxia conditions for 48 hours. At the end of the treatment (hypoxia or starvation), the cell culture medium was separated from cell debris by centrifugation (20 min, 3000 g). Then, the cell free supernatant was centrifuged for one hour at 18,000 g to obtain a pellet of microparticles. Supernatant liquid was discarded and the protein levels in the microparticle pellet were estimated using Pierce BCA Protein Assay Kit and a Nano-drop. The isolated microparticle pellet was subsequently flash frozen in liquid nitrogen in batches of 25 μg and stored at −80° C. till use.

Flow Cytometry Analysis (FACS):

Microparticle (MP) size and granularity was evaluated by flow cytometer CyAn ADP analyzer (Beckman Coulter). Forward scatter (FSC) and side scatter (SSC) parameters were set on logarithmic scales. The MP size was evaluated using standard Megamix (Biocytex, Marsille, France) and 0.78 μm beads (BD Biosciences). MP concentrations were measured using 7.5 μm count beads (Flow Cytometer Absolute Count Standard, Bangs Laboratories Inc., IN, USA). MPs were labeled for 30 minutes with antibodies (mouse anti human HLAG-1, HLAG-9, HLAG-11 and HLAG-233, followed by labeling with the secondary antibody, anti-mouse IgG Phycoerythrin (PE) for 30 minutes. Finally samples were suspended in PBS containing 0.05% formaldehyde and scanned by CyAn ADP analyzer.

Protein Array:

Purified MPs were re-suspended in “lysis buffer” (20 mM Tris pH=6.8, 150 mM NaCl, 1 mM DTT, 10% glycerol and 1% tryton (final pH=7.5)). MP protein extract was obtained from a pool of 4 specimens within each study group and quantified using the BCA protein quantification kit (Thermo Fisher Scientific Inc., Illinois USA). The Human Angiogenesis Protein Antibody Array (Ray Bio, Georgia, USA) was performed according to the manufacturer's instructions. The protein content of trophoblast MPs was normalized to the content of MPs originated from myeloma cell line. The content of circulating MPs obtained from healthy pregnant women (which contains ˜10% trophoblast MPs) was normalized to the content of circulating MPs obtained from non-pregnant women.

Migration Assay:

Endothelial cell migration was measured using 24-transwell inserts (BD Biosciences) coated with Poly-L-Lysine. HUVEC were added to the upper chamber, while MPs (trophoblast or plasma derived) in serum-free medium were added to the lower chamber (medium without MPs was used as control). After 24 hours, the inserts were fixed with 4% formaldehyde and stained with 0.5% crystal violet for 10 minutes each. Cells on the top of the membrane were removed and remaining cells on the bottom side of the membrane were photographed using inverted microscopy. The area occupied by migratory cells was calculated using the image J software.

In Vitro Wound Healing Assay:

Wound healing assay was used to evaluate the effects of trophoblast MPs on endothelial migration into “wound fields”. HUVEC were seeded in 24-well plate containing an insert that prevent cell attachment along a space of 0.9 mm area also termed “wound field”. Cells were cultured until a monolayer was formed, whereafter the insert was removed. Cell migration into the wound field was evaluated upon addition of trophoblast MPs to the culture medium. Migration in the absence of MPs served as control. Cells were photographed using inverted microscopy. The wound field area occupied by migratory cells was calculated using the image J software.

Tube Formation Assay:

HUVEC isolated from umbilical cords obtained at term of normal pregnancy were seeded on matrigel (Sigma-Aldrich, Rehovot, Israel) in 48-well tissue culture plates. MPs were added to the wells for 20 hours. The wells were continuously photographed, using time lapse imaging (Zeiss, Standort Gottingen, Germany). The length of formed tubes was measured using Image-Pro Plus software (Leeds Precision Instruments, Minneapolis, Minn.) and normalized to the initial cell number in each well.

TUNELL Assay:

Neonatal rat heart cardiomyocytes were seeded in 24-well tissue culture plates for 20 h with or without trophoblast derived MPs and cultured either under starvation conditions (serum and growth factor free medium) or in complete medium (medium containing fetal calf serum (FCS) and growth factors). The cells were then washed, and TUNELL assay was performed according to the manufacturer's instructions (Roche Diagnostics, Mannheim, Germany). Cells treated with 50 units of DNAase (Sigma-Aldrich, Israel) for 10 minutes served as a positive control. Results were expressed as percentage of TUNELL positive cells out of the total cell population in each well.

Caspase 3/7 Activity:

HUVEC were seeded in 48-well tissue culture plates and trophoblast derived MPs were added for 20 h incubation. Cells were then washed and caspase 3/7 activity assay was carried out according to the manufacturer's instructions (CASPAS 3&7 FLICA KIT, ImmunoChemistry Technology MN, USA). Cells treated with 1 uM Staurosporine served as a positive control. Results were expressed as percentage of active caspase 3 positive cells out of the total cell population in each well.

Bcl-2 mRNA expression: Cells were seeded in 24-well tissue culture plates and cultured with or without trophoblast derived MPs isolated from healthy pregnant women for 6 hours. Total RNA was extracted with a TRI-Reagent kit (Molecular Research Center, Cincinnati, Ohio, USA). RNA concentration and purity were determined by UV absorption at 260 nm and 280 nm (Nano Drop, USA). cDNA was constructed with the cDNA synthesis kit (Applied Biosystem, USA). mRNA expression of Bcl-2 was evaluated with ABI 7700TM quantitative real time PCR system (Applied Biosystems) and compared to the human housekeeping glyceraldehyde-3-phosphate-dehydrogenase (GAPDH) gene. All reagents were obtained from Applied Biosystems.

In Vivo Wound Healing and Revascularization Assay:

Adult male Sprague-Dawley rats (250-300 g; 8-weeks old) were purchased from Harlan (Jerusalem, Israel) and housed in pairs in cages in a room with an artificial 12-h light/dark cycle at a constant temperature range (24±2 1C) and relative humidity (55±10%). The rats were acclimated for 1 week before the study and had free access to standard laboratory chow and water. Rats were divided to 2 main subgroups: healthy and diabetic rats. All experimental procedures were reviewed and approved by the Technion Animal Care and Use Committee (IL-100-08-2013).

Diabetes was induced by 1-3 intraperitoneal injections of 60 mg/kg of streptozotocin. Five days after the streptozotocin injection, the blood glucose level was measured in a drop of tail blood from each streptozotocin-injected rat, using a glucometer (FreeStyle, Abbott Diabetes Care, Alameda, Calif.). Rats were considered diabetic when their blood glucose levels were higher than 300 mg per 100 ml. Healthy rats were used as the vehicle-treated non-diabetic control group.

Anesthesia was induced using an isofluorane induction box and was maintained with 2% isoflourane and oxygen Immediately after induction of anesthesia the rats were injected with buprenorphine 0.03 mg\kg subcutaneously. Skin wounds were made in anesthetized non-diabetic and diabetic rats. For the diabetic rats, these skin wounds were made 10 days after streptozotocin administration, and the presence of hyperglycemia was confirmed. Once anesthetized, the dorsal skin on each side (left and right) of the vertebral column of each rat was shaved, and then cleaned before creating two 3-cm² full-thickness skin wounds using an aseptic technique.

On the day of wounding, the rats were either control treated with 0.5 ml of low glucose DMEM cell culture medium (vehicle) or with HVT-MPs in low glucose DMEM cell culture medium. The HVT-MPs were isolated from starved trophoblast cells that were grown till passage 15 and stored in aliquots at −80° C. till the day of treatment.

Upon treatment, the wounds were covered with a dressing film—Melolin (smith&nephew medical limited) to protect the wound during self-grooming Treatments and bandaging with the dressing film were repeated every 2 days for 12 days under ketamine/isoflourane anesthesia.

The size of the open wound was determined every 2-3 days when the wounds were treated and the bandage was replaced. To this end, transparent paper was placed over each wound, and the shape of the wound was drawn on the paper. The transparent paper was then scanned and the surface of the wound area was measured by Image Analysis Image-Pro Software. The percentage of wound closure was calculated using the following formulae:

$$\% non \text{-} closed wound area = \frac{Wound area on day X}{Wound area on day zero} \times 100$$

After measuring the area of the wound on day 15, the rats were sacrificed by inhalation of carbon dioxide CO₂. Wound samples were cut from center and from margin for pathologic examination. Full-skin thickness samples of wound tissue from all the dead animals were carefully excised from the wound using a scalpel to avoid damaging its healing edge, and were immediately fixed in 10% neutral-buffered formalin. Paraffin blocks were prepared and histological analysis was performed. Expression levels of CD34 antigen, a marker for endothelial cells of blood vessels that indicates microvascular density (MVD) in healing wounds, CD68 antigen, a marker for neutrophils involved in the wound healing process and collagen fiber deposition (masson's trichrome stain (TRI)) used to differentiate between granulation tissue/matrix formation and smooth muscle, were assessed.

### Example 2

### Trophoblast MPs Characterization

In order to establish that trophoblast MPs are non-immunogenic and therefore suitable for treatment of ischemic tissue and wounds, HLAG expression on trophoblast cells and trophoblast derived microparticles (MP) was determined

To ensure that the MPs are derived from trophoblast cells, the content of human placental lactogen (hPL), uniquely expressed by trophoblast cells, was evaluated. Only cell cultures with more than 90% of cells labeled with hPL were used to produce MPs (FIG. 1).

As shown in FIG. 1, MPs obtained from stimulated trophoblast cells culture exposed HLAG-1, HLAG-9, HLA-G11 and HLA-G233 as determined by FACS. HLAG antigens are trophoblast-specific tolerogenic molecules known to inhibit maternal immune responses to paternal antigens.

### Example 3

### Protein Expression Profiles of MP's

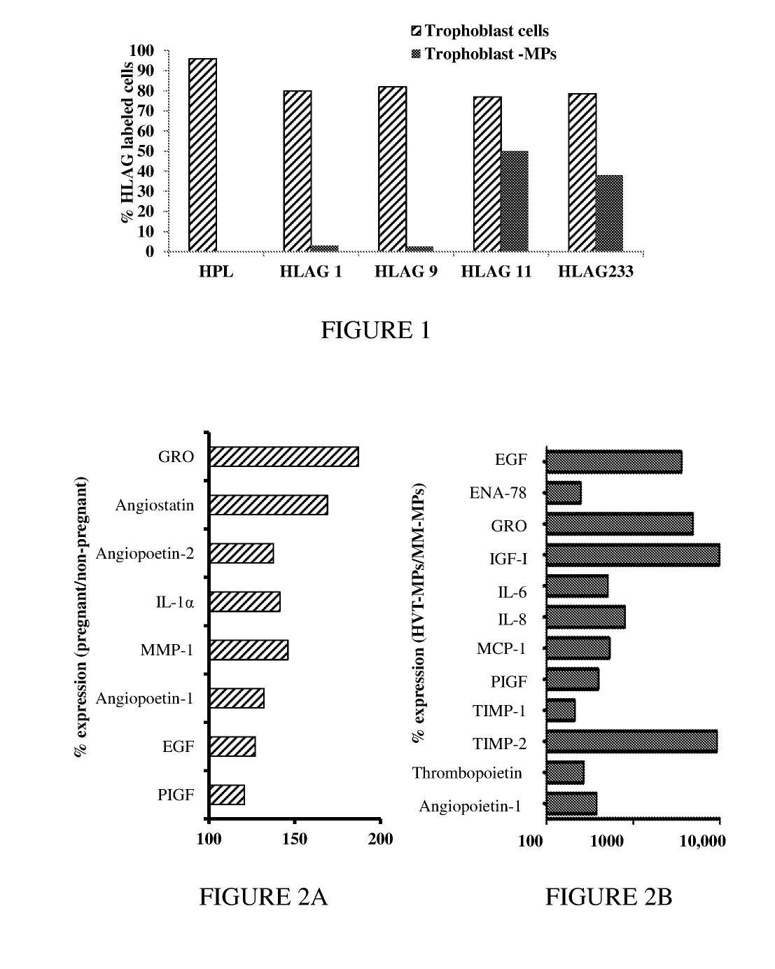

The main difference in MP population between pregnant and non-pregnant women is related to the unique presence of placental trophoblast MPs in maternal circulation of pregnant women. To identify the expression profile of MPs two comparative screens were performed. The first screen a protein array identifying the relative expression of key markers of angiogenic and inflammatory proteins in MPs obtained from non-pregnant women (NP) and healthy pregnant women (HP) (FIG. 2A). The second screen a protein array identifying the relative expression of key markers of angiogenic and inflammatory proteins in MPs obtained from trophoblast derived MPs (HVT-MPs) and MPs derived from a myeloma cell line (MM-MPs) (FIG. 2B). Surprisingly, trophoblast derived MPs had an EGF content 10 times higher than that of MP's isolated from healthy pregnant women.

The results indicate that the growth factor and cytokine composition of MPs derived from trophoblast cells (HVT-MPs) is significantly more concentrated than MPs isolated from healthy pregnant women. For example, as apparent from FIG. 2, trophoblast derived MPs expressed >7 times the level of EGF as healthy pregnant MPs. Higher levels of growth factors and cytokines such as PLGF, IGF-I and IL8 and tissue inhibitors of metalloproteinases (TIMPs) was also observed.

### Example 4

### MP Effects on Endothelial Cell Migration

To further establish the potential therapeutic effect of MP's, the effects of exposing endothelial cells to MPs was evaluated in a cell migration assay.

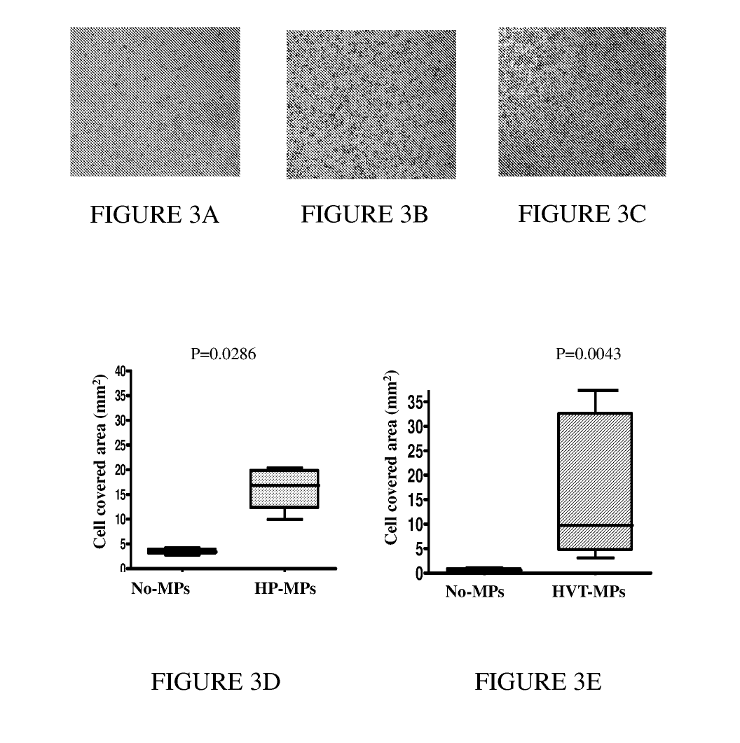

Migration was measured using 24-multiwell inserts incorporating a polyethylene terephthalate (PET) membrane that divide the chamber into an upper and a lower portion (BD Biosciences). HUVEC were added to the upper chamber of the insert while MPs (pellet of 1 mL of PPP from individual samples) in serum-free medium were added to the lower chamber (medium without MPs was used as control). After 24 hours, the inserts were fixed and stained with 0.5% crystal violet. Cells on the top of the membrane were removed and cells on the bottom side of the membrane were photographed using inverted microscopy. Exemplary images obtained from non-treated cells, cells treated with MPs obtained from healthy pregnant women and cells treated with trophoblast derived MPs (HVT-MPs) are shown in FIGS. 3A, 3B and 3C, respectively.

Total area occupied by migratory cells was calculated using the image J software. The results demonstrated that exposure of endothelial cells to circulating MPs obtained from healthy pregnant women (having ˜10% trophoblast MPs), induce a significantly elevated cell migration as compared to non-treated cells (FIG. 3D; p=0.0286). Surprisingly, exposing the cells to MPs, produced in-vitro by starved trophoblast cells, resulted in an elevated migration in comparison to non-treated cells (FIG. 3E; p=0.0043) and in comparison to cells exposed to MPs obtained from healthy pregnant women (compare FIGS. 3D and 3E).

### Example 5

### MP Effect on Wound Healing (In Vitro)

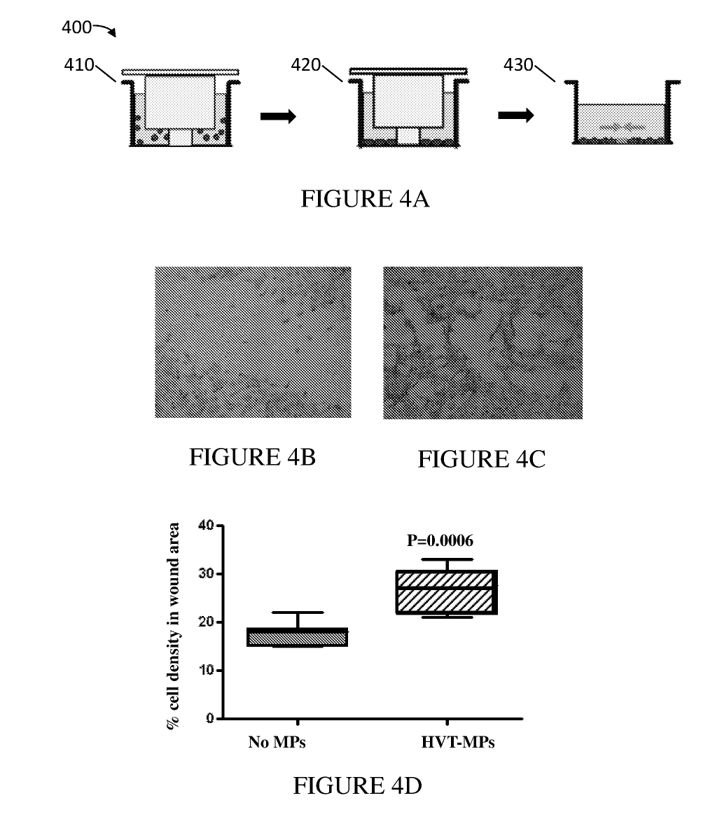

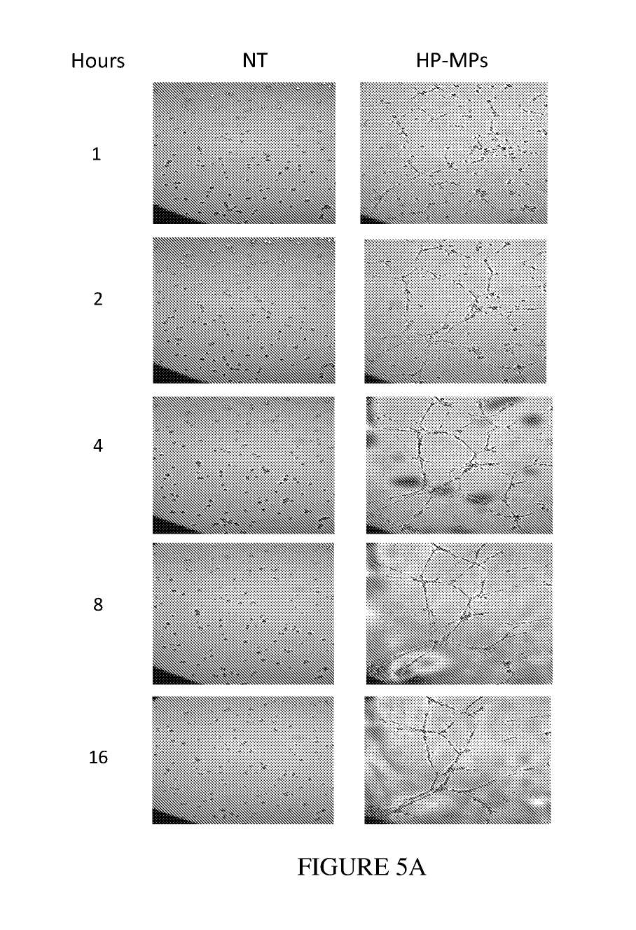

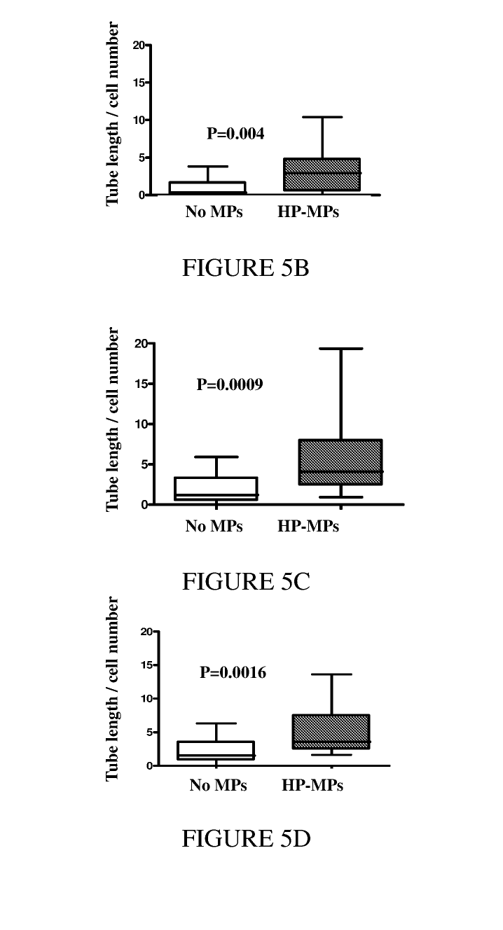

To evaluate the effects of trophoblast derived MPs on wound healing, an in vitro wound healing model, as illustrated in FIG. 4A, was used. At step 410 HUVEC were grown in 24-well plates comprising a wound healing insert. Upon generation of a monolayer, as in step 420, a 0.9 mm wound field was generated by removing the insert, as in step 430. Migration of the endothelial cells, either non-treated or exposed to trophoblast derived MPs, into the wound field was monitored for 24 h.

Exemplary images obtained from non-treated cells and cells treated with 25 μg MPs obtained from trophoblast derived MPs (HVT-MPs) are shown in FIGS. 4B and 4C, respectively and quantitatively summarized in FIG. 4D.

The results indicate that exposure of the endothelial cells to trophoblast derived MPs significantly elevates cell density in an in-vitro created wound field, as compared to non-treated cells (FIG. 4D; p=0.0006), thereby suggesting that trophoblast derived MPs enhance wound healing.

### Example 6

### MP Effects on Endothelial Tube Formation

To evaluate the ability of MPs to induce endothelial cell migration, HUVEC were seeded on matrigel (Sigma-Aldrich, Rehovot, Israel) in 48-well tissue culture plates with serum and growth factor free medium. MPs were obtained from 1 ml of plasma isolated from individual samples (circulating MPs containing ˜10% of trophoblast MPs (HP-MPs)). The wells were either treated by adding pellet of HP-MPs obtained from 1 mL of PPP from individual samples of healthy pregnant women in serum-free medium to the wells for 20 hours or left untreated. The wells were continuously photographed, using time lapse imaging (Zeiss, Standort Gottingen, Germany). Exemplary images obtained from non-treated cells (left panel) and cells treated with the HP-MPs (right panels) at the indicated hours are shown in FIG. 5A.

The length of formed tubes was measured using Image-Pro Plus software (Leeds Precision Instruments, Minneapolis, Minn.) and normalized to the initial cell number in each well and then compared to the tube lengths obtained in cells to which MPs were not added. The results demonstrated that exposure of HUVEC grown in starvation conditions to HP-MPs, induce enhanced branched tube network formation within one hour as compared to cells in serum-free medium only (FIG. 5B-5D).

In summary, the tube formation data indicate that exposure of the endothelial cells to trophoblast derived MPs significantly elevates the ability of HUVEC to form branched tube network in-vitro as compared to non-treated cells, after 1 hr (p=0.004); 2 hr (p=0.0009) or 4 hr (p=0.0016) incubation with the aforementioned MPs. The results clearly suggest that trophoblast derived MPs induce wound healing.

### Example 7

### Trophoblast MPs Affect Cell Survival

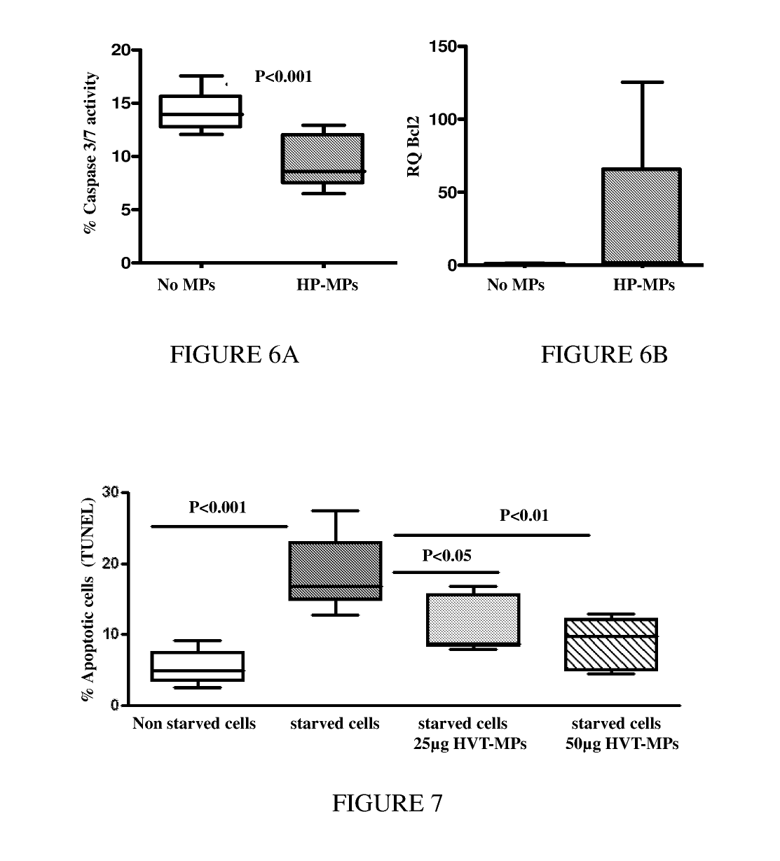

The survival and apoptosis of endothelial cells upon exposure to MPs was evaluated. HUVEC were exposed to MPs obtained from 1 ml of plasma, isolated from individual samples of healthy pregnant women (e.g. circulating MPs containing ˜10% trophoblast MPs (HP-MPs)). Apoptosis was evaluated using caspase 3/7 activity assay, as elaborated herein above. The results demonstrate that exposure of HUVEC to MPs (HP-MPs; n=9) derived from healthy pregnant women significantly decrease caspase 3/7 activity as compared to untreated (No MPs; n=9) cells (FIG. 6A; p<0.001).

Furthermore, the effect of exposing cells to HP-MPs on cell expression of the anti-apoptotic gene, Bcl-2 was evaluated. HUVEC were cultured with (HP-MPs; n=8) or without (No MPs; n=3) MPs obtained from 1 mL of plasma of healthy pregnant women and Bcl-2 mRNA expression was evaluated by real time PCR. As seen in FIG. 6B, exposure of the HUVEC to HP-MPs induced expression of Bcl-2].

To assess the ability of human trophoblast derived MPs to improve survival of heart cells, 25 μg or 50 μg of trophoblast cell derived MPs (HVT-MPs) were added to rat cardiomyocytes grown under starvation conditions. Apoptotic rate of these cells was evaluated by TUNELL assay and compared to non-starved cells or untreated starved cells. As seen in FIG. 7, trophoblast derived MPs significantly (p<0.05) reduced cardiomyocyte apoptosis in a dose-dependent manner.

### Example 8

### MP Effect on Wound Healing In Vivo—Healthy Rats

Full-thickness skin wounds were made in two rats. The wound of the first rat served as control (i.e. treated with vehicle) and the left and right side wounds of the second rat were treated with 50 μg and 100 μg human trophoblast derived MPs (HVT-MPs), respectively. Wound healing (wound closure) was quantitatively assessed by determining the wound size on days 1 (day of wounding and first treatment), 3, 6, 10, 13 and 15. Results obtained for the rat treated with the HVT-MPs were compared to those obtained for the vehicle treated rat.

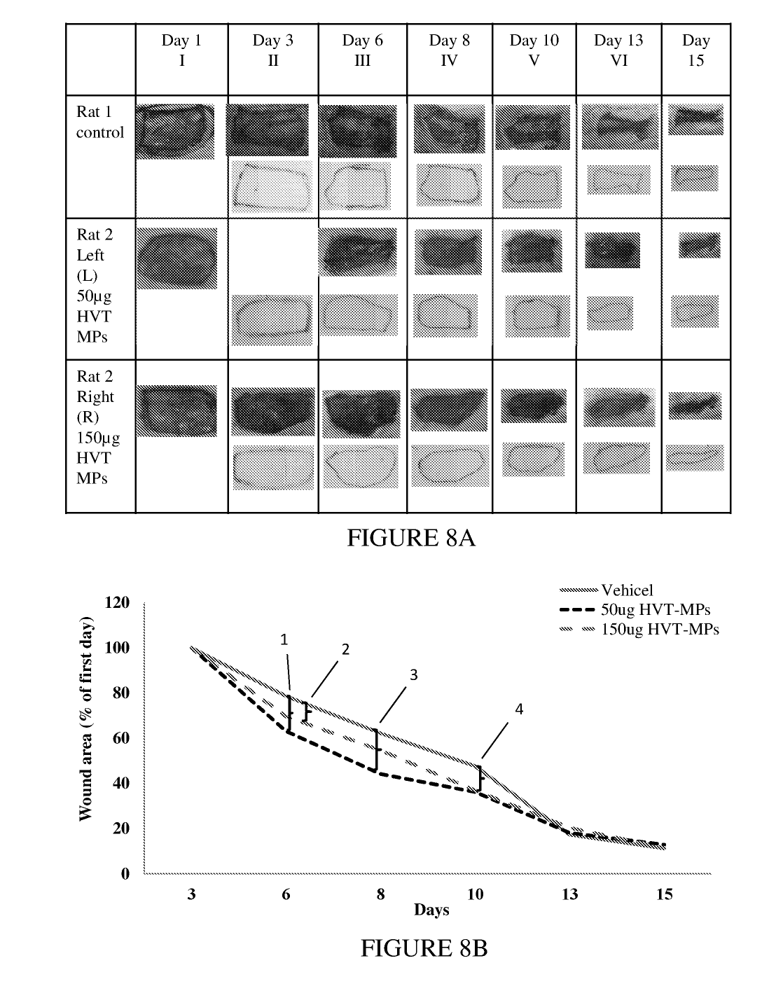

Exemplary images of non-treated wounds (upper panel), wounds treated with 50 μg HVT-MPs (center panel), and wounds treated with 100 μg HVT-MPs (lower panel) are shown in FIG. 8A. FIG. 8B represents the wound area as a function of time. As seen from the figures, from day 3 up to day 10, the areas of the HVT-MPs-treated wounds were significantly smaller than the corresponding vehicle treated wounds. A dose of 50 μg HVT-MPs (black broken line) reduced the wound area by 20%, after 6 days of treatment and by 29% after 8 days of treatment (indicated by the numbers 1 and 3 in FIG. 8B), as compared to the non-treated wounds (grey line). A higher dose of 150 μg HVT-MPs (grey broken line) reduced the wound area by 12% after 6 days, as compared to the non-treated wounds (indicated by the number 2 in FIG. 8B). The two doses of HVT-MPs reduced the wound area by 24% after 10 days of treatment, as compared to the non-treated wounds (indicated by the number 4 in FIG. 8B).

### Example 9

### MP Effect on Wound Healing In Vivo—Diabetic Rats

Trophoblast derived MPs (HVT-MPs) were used for wound healing in a rat diabetic wound model. Diabetes was induced in three adult male Sprague—Dawley rats (250-300 g; 8-weeks old), as essentially described above.

Two full-thickness skin wound were induced in each side of the rats. The right-side wounds of the first rat was treated with 250 μg HVT-MPs (Rat I). The right side wound of the second rat was treated with 500 μg HVT-MP (Rat II). The right side wound of the second rat was control (vehicle) treated (Rat III). The left side wounds of all rats were left untreated and served as control.

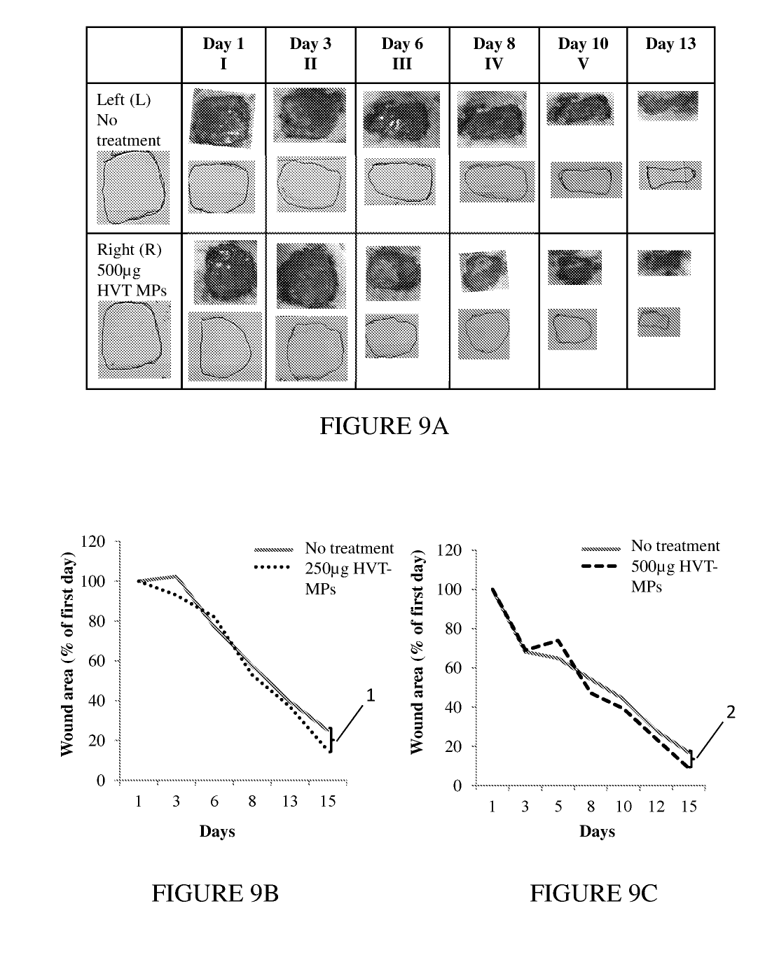

Exemplary images of the left side non-treated wound of Rat II (upper panel) and the right sided wound of Rat II treated with 500 μg HVT-MPs (lower panel) are shown in FIG. 9A. FIGS. 9B and 9C show a quantification of the wound area size as a function of time for Rat I treated with 250 μg HVT-MPs (dotted line, FIG. 9B) and Rat II treated with 500 μg HVT-MP (broken line, FIG. 9C), as compared to untreated wounds (grey line). As seen in FIG. 9B, treating the wounds with 250 μg (Rat I) resulted in a 40% reduction in the wound area (indicated by the number 1 in FIG. 9B), as compared to the non-treated wounds, at day 15 of the study. Similarly, as seen in FIG. 9C, treating the wounds with 500 μg (Rat II) resulted in a 48% reduction in the wound area (indicated by the number 2 in FIG. 9C), as compared to the non-treated wounds, at day 15 of the study.

On day 15, the animals were sacrificed and tissue samples from all rats were taken from the center and from margin of the wounds. The samples underwent pathologic examination for the expression level of CD34, CD68 and collagen. CD34 is a marker for endothelial cells of blood vessels and thus serves as an indication of the microvascular density (MVD) of the wounds. CD68 is a neutrophil cell marker involved in the wound healing process. CD34 and CD68 serve as markers of early stage wound healing. Collagen deposition represents an advanced stage of wound healing.

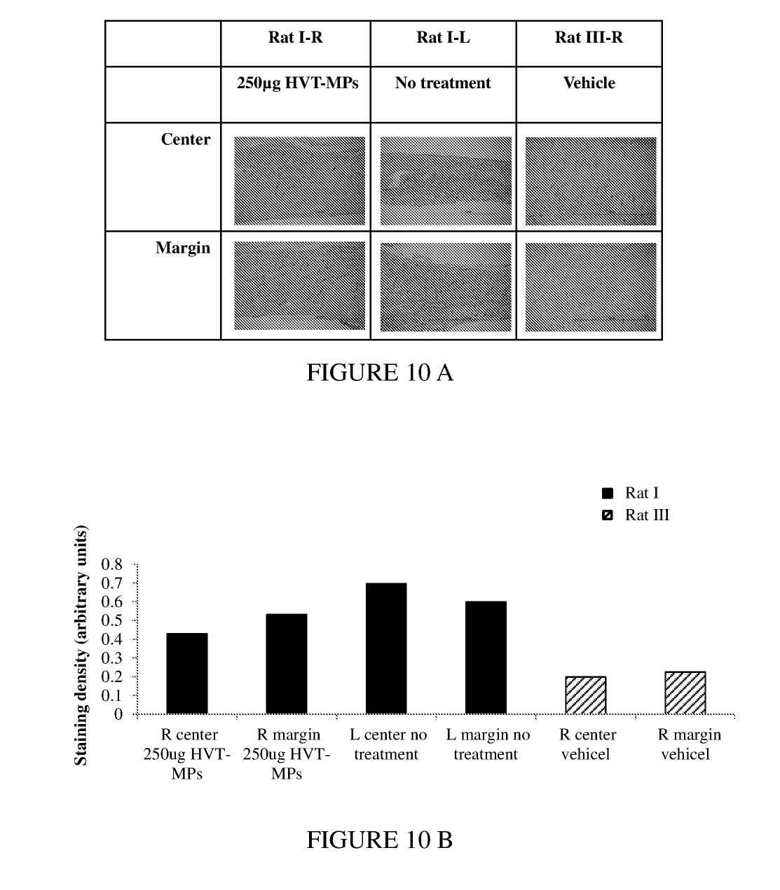

Exemplary images tissue sections stained for CD34, CD68 and Collagen are shown in FIGS. 10A, 11A and 12A respectively. Wound samples taken from the center and margin of the wounds (upper and lower panels, respectively) of the left side non-treated wound of Rat I, from the right side wound of Rat I treated with 250 μg HVT-MPs and from the right side vehicle treated wound of Rat III are shown.

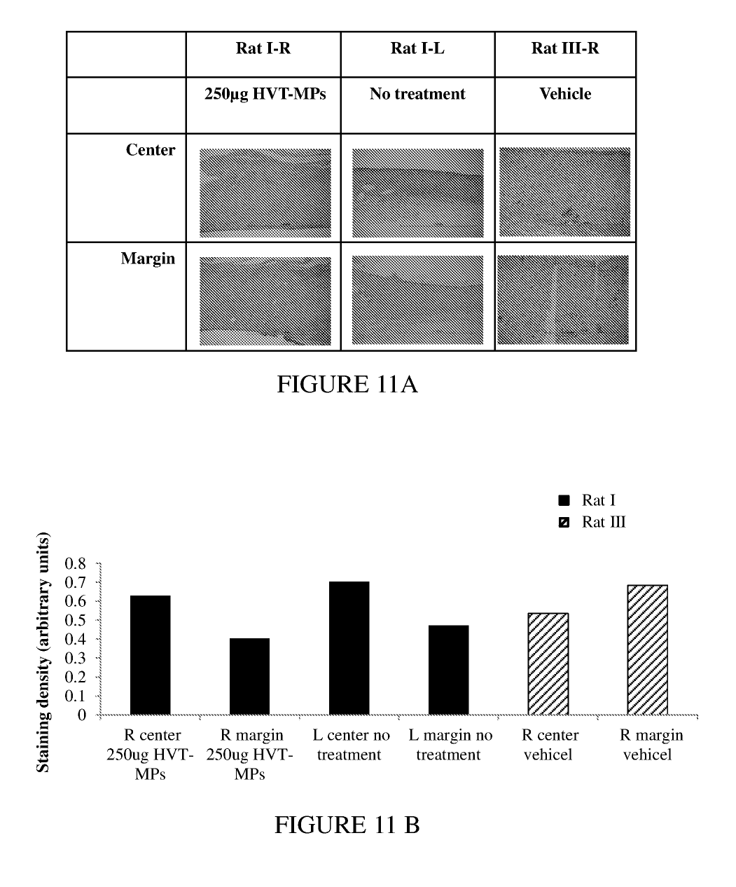

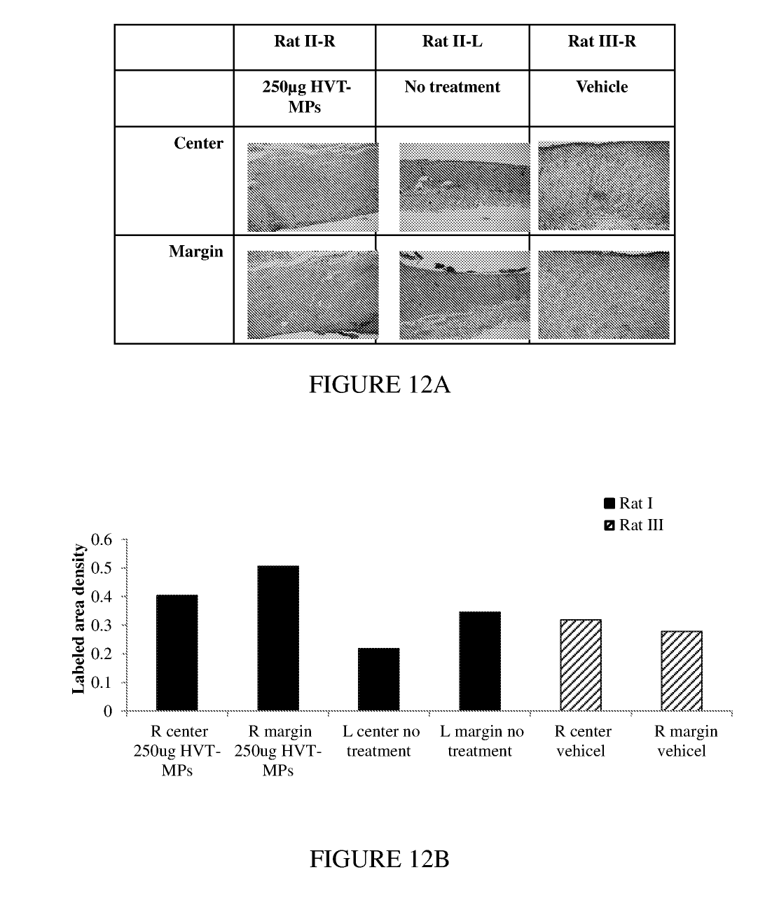

FIGS. 10B, 11B and 12B show a quantification of CD34, CD68 and Collagen staining density, respectively. Wounds treated with 250 μg HVT-MPs were characterized by a higher collagen density (FIG. 12B), and lower levels of CD34 staining (FIG. 10B) and CD68 staining (FIG. 11B). The results resemble an expression pattern typical for an advanced stage of wound healing. In contrast, the untreated wounds were characterized by a lower collagen density, and higher level of CD34 and CD68 staining, as typical for early stages of wound healing.

### Example 10

### MP Effect on Wound Healing In Vivo

Trophoblast-MPs are used for wound healing and revascularization of ischemic tissue in an additional rat myocardial infarction animal model for evaluation of trophoblast MP effect on myocardial revascularization.

To perform myocardial infarction, rats are anesthetized and a left thoracotomy is performed in the fourth intercostal space. The pericardium is opened to expose the heart. For ligation a 7-0 silk suture is passed around a prominent branch of the left coronary artery with a taper needle. A pale and akinetic region is delineated on the surface of the middle to apical portion of the left ventricle, corresponding to the area of severe ischemia. Four injections of trophoblast MPs (250 μg/ml protein) in PBS (20 μl per injection) are performed at the infracted area. Sham-operated rats, injected with PBS only, into the same region of the ischemic heart muscle, and serve as control. In addition, the effect of co-transplantation of cardiomyocytes and trophoblast derived MPs on infarcted heart is evaluated. Infarcted heart transplanted with cardiomyocytes or trophoblast derived MPs alone serve as control. Cardiac function is assessed by echocardiography as well as by histology to evaluate infarct size, tissue vascularization, tissue perfusion, cell graft survival and integration, and cardiac remodeling.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the spirit of the invention.

1-28. (canceled)

29. A method of treating damaged tissue in a subject in need thereof, comprising administering to the subject a pharmaceutical composition comprising a therapeutically effective amount of microparticles derived from trophoblast cells.

30. (canceled)

31. The method of claim 29, wherein the wound is a chronic wound.

32. The method of claim 29, wherein the damaged tissue is ischemic tissue.

33. The method of claim 29, wherein the trophoblast cells are early-stage trophoblast cells.

34. The method of claim 29, wherein the trophoblast cells comprise an in vitro grown trophoblast cell culture.

35. The method of claim 34, wherein the microparticles are obtained from the in vitro trophoblast cell culture.

36. The method of claim 35, further comprising culturing the trophoblast cell culture under starvation condition prior to obtaining said microparticles.

37. The method of claim 29, wherein the microparticles comprise membrane vesicles.

38. The method of claim 29, wherein the microparticles comprise non-classical HLAG.

39. The method of claim 29, wherein the microparticles comprise growth factors.

40. The method of claim 29, wherein the microparticles comprise epidermal growth factor.

41. The method of claim 29, wherein the microparticles comprise cytokines.

42. The method of claim 29, wherein the microparticles are derived from trophoblast cells isolated from the plasma of pregnant subjects.

43. The method of claim 29, wherein the subject in need thereof is diabetic.

44. The method of claim 29, wherein the pharmaceutical composition is administered topically.

45. A pharmaceutical composition comprising a therapeutically effective amount of microparticles derived from trophoblasts for treating damaged tissue, wherein said microparticles are isolated from a trophoblast cell culture.

46. The pharmaceutical composition of claim 45, wherein prior to isolating said microparticles, the trophoblast cell culture is exposed to a stress condition.

47. The pharmaceutical composition of claim 46, wherein the stress condition is selected from starvation, hypoxia or a combination thereof.
